(19) United States
(12) Patent Application Publication
(10) Pub. No.: US 2019/0136192 A1
(43) Pub. Date: May 9, 2019

(54) MESENCHYMAL STEM CELL THERAPY FOR SPINAL MUSCULAR ATROPHY

(71) Applicant: CELL MEDICINE, INC., Farmers Branch, TX (US)

(72) Inventor: Neil RIORDAN, Westlake, TX (US)

(73) Assignee: CELL MEDICINE, INC., FARMERS BRANCH, TX (US)

(21) Appl. No.: 16/185,055

(22) Filed: Nov. 9, 2018

Related U.S. Application Data
(60) Provisional application No. 62/584,001, filed on Nov. 9, 2017.

(51) Int. Cl. C12N 5/0775; A61K 35/28; A61P 21/00; A61P 29/00; A61K 9/00; C12N 5/073; C12N 5/071

(52) U.S. Cl. CPC C12N 5/0665; A61K 35/28; A61P 21/00; A61P 29/00; C12N 5/0682; C12N 5/0663; C12N 5/0667; C12N 5/0668; C12N 5/0605; A61K 9/0019

(57) ABSTRACT

Disclosed are means, methods and compositions of matter useful for treatment of spinal muscular atrophy. In one embodiment, stem cells of the mesenchymal type are modified to enhance anti-inflammatory and regenerative potential in a manner to prevent disease, inhibit progression and/or reverse existing disease. In other embodiments combinations of mesenchymal stem cells together with extracts and/or products derived from said mesenchymal stem cells are administered for prevention, inhibition of progression and/or reversion of spinal muscular atrophy.

### CROSS-REFERENCE TO RELATED APPLICATIONS

This application claims the benefit of priority to U.S. Provisional Application No. 62/584,001, filed Nov. 9, 2017, the contents of which are incorporated herein by reference.

### FIELD OF THE INVENTION

The invention pertains to the treatment of genetic diseases. More specifically, the invention parties t the field of spinal muscular atrophy, more specifically, the invention pertains to administration of stem cells to prevent and/or reduce pathology of this condition by stem cell administration alone and/or in combination with derivatives of stem cells, and/or other treatments associated with increasing production of the SMN protein.

### BACKGROUND OF THE INVENTION

Spinal muscular atrophy (SMA) is neurodegenerative disease that is one of the most frequent genetic cause of infant mortality [1], affecting approximately 1 in 6,000-10,000 individuals world-wide, with 1 in 60 individuals being carriers [2]. SMA is inherited in an autosomal recessive manner caused at a molecular level by the homozygous deletion or mutation of the survival of motor neuron 1 (SMN1) gene, which results in a deficiency of the ubiquitously expressed SMN protein. SMN2, is a gene unique to humans, is an almost identical copy gene of SMN1, but has a constitutive C to T transition in its exon 7. This transition affects the splicing of SMN2 mRNA, thereby resulting in the predominant production of a shorter unstable isoform termed SMN-Δ7 [3]. Although SMN2 is unable to compensate for the homozygous loss of SMN1 because of the lower amount of full-length SMN transcripts (SMN-FL), the copy number of SMN2 affects the severity of SMA [4], correlating with the different grades of SMA severity [5-7]. Functionally, the loss of active SMN levels results in degeneration of alpha motor neurons of the spinal cord and resulting in muscle weakness and progressive symmetrical proximal paralysis [8].

There are 4 types of SMA, graded based on severity and onset. SMA-I, also known as Werdnig-Hoffmann disease, is the most devastating and also the most prevalent form of the disease, comprising approximately 50% of SMA patients. Patients with SMA-1 generally display clinical characteristics before 6 months of age and typically perish before age of 2 [9]. Diagnosis of SMA-II occurs between 7 and 18 months of age. Patients achieve the ability to sit erect unsupported, with a proportion of them having ability to stand erect. In these patients, deep tendon reflexes cannot be found and microtremors of upper extremities are common. Joint contractures and kyphoscoliosis are very common and can occur in the first years of life in the more severe type II patients [10]. SMA-III patients, also termed Kugelberg-Welander disease, usually achieve all major motor developmental milestones, including walking. During infancy these patients develop proximal muscular weakness. A heterogeneity of clinical presentation is noticed in which some require wheelchair assistance in childhood, whereas others might continue to walk and live productive adult lives with minor muscular weakness [11].

One of the most prominent features of SMA is mutation or loss of the spinal motor neuron (SMN) gene. The biological functions of SMN are diverse and cell-type specific. Studies have shown that the SMN protein is conserved across various lifeforms and ubiquitously expressed. Cellular localization of SMN is found in the cytoplasm and the nucleus. In the nucleus SMN has been shown to concentrate in areas that are similar in number (2-6) and size (0.1-1.0 micron) to coiled bodies, and frequently are found near or associated with coiled bodies. Liu and Dreyfuss, who identified these dense intranuclear bodies containing SMN termed them “Gems”, based on Gemini of coiled bodies [12]. Subsequently researchers found that the Gems structures are associated with Cajal bodies, which are nuclear domains implicated in the formation and shaping of ribonucleoprotein complexes RNPs. This finding led investigators to identify SMN as playing a role in RNA regulation [13]. Further studies have demonstrated that SMN associates the proteins Gemins 2-8 and Unrip to form a large macromolecular complex through a network of reciprocal interactions [14]. This macromolecule complex is essential for formation RNPs, splicing, transcription and axonal mRNA transport, all of which are impaired in SMA, and thereby resulting in pathology [15]. Indeed efforts are underway to develop pharmaceutics that alleviate the neuromuscular phenotype by restoring the fundamental function of SMN in formation of the SMN-Gemins complex without necessarily augmenting SMN levels [16]. One example of treating SMA without necessarily augmenting levels of SMN was a paper by Oprea et al, who observed that in some cases of SMN deficiencies, normal axonogenesis was evident in cases where plastin-3 was overexpressed. They found that SMA mouse and zebrafish embryos, the forced overexpression of plastin-3 rescued axonogenesis in part through augmentation of F-actin levels [17].

Currently patients with SMA are treated by nutritional and respiratory care, as well as physiotherapy. These are only useful in maintaining some quality of life but do not impact the underlying disease process [8, 18]. Clinical trials have been conducted with pharmacological agents such as valproic acid [19], phenylbutyrate [20], and hydroxyurea [21], however no benefit in randomized double blind trials was observed. Unfortunately, although tendency of improvement was seen in earlier trials [22-25], these have not been reproduced, even these tendency of improvements were marginal. Thus novel approaches are needed in the treatment of SMA.

### SUMMARY

Certain embodiments herein are directed to methods wherein said mesenchymal stem cells are derived from tissues selected from a group comprising of: a) bone marrow; b) peripheral blood; c) adipose tissue; d) mobilized peripheral blood; e) umbilical cord blood; f) Wharton's jelly; g) umbilical cord tissue; h) skeletal muscle tissue; i) subepithelial umbilical cord; j) endometrial tissue; k) menstrual blood; and 1) fallopian tube tissue.

Certain embodiments herein are directed to methods wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Certain embodiments herein are directed to methods wherein said mesenchymal stem cells from umbilical cord tissue do not express markers selected from a group comprising of: a) CD117; b) CD31; c) CD34; and CD45;

Certain embodiments herein are directed to methods wherein said mesenchymal stem cells from umbilical cord tissue express, relative to a human fibroblast, increased levels of interleukin 8 and reticulon 1

Certain embodiments herein are directed to methods wherein said mesenchymal stem cells from umbilical cord tissue have the potential to differentiate into cells of at least a skeletal muscle, vascular smooth muscle, pericyte or vascular endothelium phenotype.

Certain embodiments herein are directed to methods wherein said mesenchymal stem cells from umbilical cord tissue express markers selected from a group comprising of: a) CD10; b) CD13; c) CD44; d) CD73; and e) CD90.

Certain embodiments herein are directed to methods wherein said umbilical cord tissue mesenchymal stem cell is an isolated umbilical cord tissue cell isolated from umbilical cord tissue substantially free of blood that is capable of self-renewal and expansion in culture,

Certain embodiments herein are directed to methods wherein said umbilical cord tissue mesenchymal stem cells has the potential to differentiate into cells of other phenotypes.

Certain embodiments herein are directed to methods wherein said other phenotypes comprise: a) osteocytic; b) adipogenic; and c) chondrogenic differentiation.

Certain embodiments herein are directed to methods wherein said cord tissue derived mesenchymal stem cells can undergo at least 20 doublings in culture.

Certain embodiments herein are directed to methods wherein said cord tissue derived mesenchymal stem cell maintains a normal karyotype upon passaging

Certain embodiments herein are directed to methods wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Certain embodiments herein are directed to methods wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Certain embodiments herein are directed to methods wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; 1) RANTES; and m) TIMP1

Certain embodiments herein are directed to methods wherein said umbilical cord tissue derived cells express markers selected from a group comprising of: a) TRA1-60; b) TRA1-81; c) SSEA3; d) SSEA4; and e) NANOG.

Certain embodiments herein are directed to methods wherein said umbilical cord tissue-derived cells are positive for alkaline phosphatase staining.

Certain embodiments herein are directed to methods wherein said umbilical cord tissue-derived cells are capable of differentiating into one or more lineages selected from a group comprising of; a) ectoderm; b) mesoderm, and; c) endoderm.

Certain embodiments herein are directed to methods wherein said bone marrow derived mesenchymal stem cells possess markers selected from a group comprising of: a) CD73; b) CD90; and c) CD105.

Certain embodiments herein are directed to methods wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; 1) 6-19; m) thrombomodulin; n) telomerase; o) CD10; p) CD13; and q) integrin beta.

Certain embodiments herein are directed to methods wherein said bone marrow derived mesenchymal stem cell is a mesenchymal stem cell progenitor cell.

Certain embodiments herein are directed to methods wherein said mesenchymal progenitor cells are a population of bone marrow mesenchymal stem cells enriched for cells containing STRO-1

Certain embodiments herein are directed to methods wherein said mesenchymal progenitor cells express both STRO-1 and VCAM-1.

Certain embodiments herein are directed to methods wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.

Certain embodiments herein are directed to methods wherein said bone marrow mesenchymal stem cells lack expression of CD14, CD34, and CD45.

Certain embodiments herein are directed to methods wherein said STRO-1 expressing cells are positive for a marker selected from a group comprising of: a) VACM-1; b) TKY-1; c) CD146 and; d) STRO-2

Certain embodiments herein are directed to methods wherein said bone marrow mesenchymal stem cell express markers selected from a group comprising of: a) CD13; b) CD34; c) CD56 and; d) CD117

Certain embodiments herein are directed to methods wherein said bone marrow mesenchymal stem cells do not express CD10.

Certain embodiments herein are directed to methods wherein said bone marrow mesenchymal stem cells do not express CD2, CD5, CD14, CD19, CD33, CD45, and DRII.

Certain embodiments herein are directed to methods wherein said bone marrow mesenchymal stem cells express CD13,CD34, CD56, CD90, CD117 and nestin, and which do not express CD2, CD3, CD10, CD14, CD16, CD31, CD33, CD45 and CD64.

Certain embodiments herein are directed to methods wherein said skeletal muscle stem cells express markers selected from a group comprising of: a) CD13; b) CD34; c) CD56 and; d) CD117

Certain embodiments herein are directed to methods wherein said skeletal muscle mesenchymal stem cells do not express CD10.

Certain embodiments herein are directed to methods wherein said skeletal muscle mesenchymal stem cells do not express CD2, CD5, CD14, CD19, CD33, CD45, and DRII.

Certain embodiments herein are directed to methods wherein said bone marrow mesenchymal stem cells express CD13,CD34, CD56, CD90, CD117 and nestin, and which do not express CD2, CD3, CD10, CD14, CD16, CD31, CD33, CD45 and CD64.

Certain embodiments herein are directed to methods wherein said subepithelial umbilical cord derived mesenchymal stem cells possess markers selected from a group comprising of; a) CD29; b) CD73; c) CD90; d) CD166; e) SSEA4; f) CD9; g) CD44; h) CD146; and i) CD105

Certain embodiments herein are directed to methods wherein said subepithelial umbilical cord derived mesenchymal stem cells do not express markers selected from a group comprising of; a)CD45; b) CD34; c) CD14; d) CD79; e) CD106; CD86; g) CD80; h) CD19; i) CD117; j) Stro-1 and k) HLA-DR.

Certain embodiments herein are directed to methods wherein said subepithelial umbilical cord derived mesenchymal stem cells express CD29, CD73, CD90, CD166, SSEA4, CD9, CD44, CD146, and CD105.

Certain embodiments herein are directed to methods wherein said subepithelial umbilical cord derived mesenchymal stem cells do not express CD45, CD34, CD14, CD79, CD106, CD86, CD80, CD19, CD117, Stro-1, and HLA-DR.

Certain embodiments herein are directed to methods wherein said subepithelial umbilical cord derived mesenchymal stem cells are positive for SOX2.

Certain embodiments herein are directed to methods wherein said subepithelial umbilical cord derived mesenchymal stem cells are positive for OCT4.

Certain embodiments herein are directed to methods wherein said subepithelial umbilical cord derived mesenchymal stem cells are positive for OCT4 and SOX2.

Certain embodiments herein are directed to methods wherein said stem cells are pluripotent stem cells.

Certain embodiments herein are directed to methods wherein said pluripotent stem cells are selected from a group comprising of: a) embryonic stem cells; b) parthenogenic derived stem cells; c) inducible pluripotent stem cells; d) somatic cell nuclear transfer derived stem cells; e) cytoplasmic transfer derived stem cells; and f) stimulus-triggered acquisition of pluripotency.

Certain embodiments herein are directed to methods wherein said stem cells are hematopoietic stem cell.

Certain embodiments herein are directed to methods wherein said hematopoietic stem cells are capable of multi-lineage reconstitution in an immunodeficient host.

Certain embodiments herein are directed to methods wherein said hematopoietic stem cells express the c-kit protein.

Certain embodiments herein are directed to methods wherein said hematopoietic stem cells express the Sca-1 protein.

Certain embodiments herein are directed to methods wherein said hematopoietic stem cells express CD34.

Certain embodiments herein are directed to methods wherein said hematopoietic stem cells express CD133.

Certain embodiments herein are directed to methods wherein said hematopoietic stem cells lack expression of lineage markers.

Certain embodiments herein are directed to methods wherein said hematopoietic stem cells lack expression of CD38.

Certain embodiments herein are directed to methods wherein said hematopoietic stem cells are positive for expression of c-kit and Sca-1 and substantially lack expression of lineage markers.

Certain embodiments herein are directed to methods wherein said hematopoietic stem cells are derived from a group of sources, said group comprising of: a) peripheral blood; b) mobilized peripheral blood; c) bone marrow; d) cord blood; e) adipose stromal vascular fraction; and f) derived from progenitor cells.

Certain embodiments herein are directed to methods wherein said derivatives of said stem cells are a stem cell conditioned media.

Certain embodiments herein are directed to methods wherein said stem cell conditioned media is a liquid media that has been exposed to a stem cell population.

Certain embodiments herein are directed to methods wherein said stem cell population that has been exposed to said liquid media are in a proliferative state at initiation of exposure.

Certain embodiments herein are directed to methods wherein said stem cells used to generate said conditioned media are treated with conditions stimulating release of growth factors capable of inducing regeneration in endogenous tissue of an SMA patient.

Certain embodiments herein are directed to methods wherein said conditions are selected from a group comprising of: a) hypoxia; b) stimulation with inflammatory mediators; c) acidosis; and d) hyperthermia.

Certain embodiments herein are directed to methods wherein said hypoxia comprises incubation of said stem cells under conditions of 02% oxygen to 15% oxygen volume by volume.

Certain embodiments herein are directed to methods wherein said inflammatory mediator is selected from a group comprising of: a) interferon gamma; b) interleukin 1; c) interleukin 6; d) TNF-alpha; e) interleukin 11; f) interleukin 17; g) interleukin 18; h) interleukin 21; i) interleukin 23; and i) interleukin 27.

Certain embodiments herein are directed to methods wherein said inflammatory mediator is intravenous immunoglobulin.

Certain embodiments herein are directed to methods wherein said inflammatory mediator is monocyte conditioned media.

Certain embodiments herein are directed to methods wherein said inflammatory mediator is supernatant of a mixed lymphocyte reaction.

Certain embodiments herein are directed to methods wherein said inflammatory mediator is a coculture of said mesenchymal stem cells with allogeneic lymphocytes.

73. The method of claim 66, wherein said inflammatory mediator is an agent capable of inducing signaling through a Pathogen Associated Molecular Pattern (PAMP) receptor.

Certain embodiments herein are directed to methods wherein said PAMP receptor is selected from a group comprising of: a) MDA5; b) RIG-1; and c) NOD.

Certain embodiments herein are directed to methods wherein said toll like receptor is TLR-2.

Certain embodiments herein are directed to methods wherein said TLR-2 is activated by compounds selected from a group comprising of: a) Pam3cys4; b) Heat Killed Listeria monocytogenes (HKLM); and c) FSL-1.

Certain embodiments herein are directed to methods wherein said toll like receptor is TLR-3.

Certain embodiments herein are directed to methods wherein said TLR-3 is activated by Poly IC.

Certain embodiments herein are directed to methods wherein said TLR-3 is activated by double stranded RNA.

Certain embodiments herein are directed to methods wherein said double stranded RNA is of mammalian origin.

Certain embodiments herein are directed to methods wherein said double stranded RNA is of prokaryotic origin.

Certain embodiments herein are directed to methods wherein said double stranded RNA is derived from leukocyte extract.

Certain embodiments herein are directed to methods wherein said leukocyte extract is a heterogeneous composition derived from freeze-thawing of leukocytes, followed by dialysis for compounds less than 15 kDa.

Certain embodiments herein are directed to methods wherein said toll like receptor is TLR-4.

Certain embodiments herein are directed to methods wherein said TLR-4 is activated by lipopolysaccharide.

Certain embodiments herein are directed to methods wherein said TLR-4 is activated by a peptide.

Certain embodiments herein are directed to methods wherein said TLR-4 is activated by HMGB-1.

Certain embodiments herein are directed to methods wherein said TLR-4 is activated by a peptide derived from HMGB-1.

Certain embodiments herein are directed to methods wherein said HMGB-1 peptide is hp91.

Certain embodiments herein are directed to methods wherein said toll like receptor is TLR-5.

Certain embodiments herein are directed to methods wherein said TLR-5 is activated by flagellin.

Certain embodiments herein are directed to methods wherein said toll like receptor is TLR-7.

Certain embodiments herein are directed to methods wherein said TLR-7 is activated by imiquimod.

Certain embodiments herein are directed to methods wherein said toll like receptor is TLR-8.

Certain embodiments herein are directed to methods wherein said TLR-8 is activated by resmiqiumod.

Certain embodiments herein are directed to methods wherein said toll like receptor is TLR-9

Certain embodiments herein are directed to methods wherein said TLR-9 is activated by CpG DNA.

Certain embodiments herein are directed to methods wherein said stem cell derived products are stem cell derived microvesicles.

Certain embodiments herein are directed to methods wherein said stem cell derived products are stem cell derived exosomes.

Certain embodiments herein are directed to methods wherein said stem cell derived products are stem cell derived apoptotic vesicles.

Certain embodiments herein are directed to methods wherein said stem cell derived products are stem cell derived miRNAs.

Certain embodiments herein are directed to methods wherein said exosomes possess a size of between 30 nm and 150 nm.

Certain embodiments herein are directed to methods wherein said exosome possesses a size of between 2 nm and 200 nm, as determined by filtration against a 0.2 .mu.M filter and concentration against a membrane with a molecular weight cut-off of 10 kDa, or a hydrodynamic radius of below 100 nm as determined by laser diffraction or dynamic light scattering.

Certain embodiments herein are directed to methods wherein said exosome possesses a lipid selected from the group consisting of: a) phospholipids; b) phosphatidyl serine; c) phosphatidyl inositol; d) phosphatidyl choline; e) sphingomyelin; f) ceramides; g) glycolipid; h) cerebroside; i) steroids, and j) cholesterol.

Certain embodiments herein are directed to methods wherein said stem cell, and/or stem cell derived product is administered prior to, and/or concurrent with, and/or subsequent to administration of an effective amount of a sodium-proton exchanger inhibitor which possesses the ability to increase the expression level of SMN exon 7 in cells of the subject.

Certain embodiments herein are directed to methods wherein said sodium-proton exchanger inhibitor is 5-(N-ethyl-N-isopropyl)-amiloride.

Certain embodiments herein are directed to methods wherein said sodium-proton exchanger inhibitor induces the expression of SRp20 protein and increases the number of nuclear gems.

Certain embodiments herein are directed to methods wherein the ratio of SMN transcripts having exon 7 to those lacking exon 7 is increased by at least 50%.

Certain embodiments herein are directed to methods wherein stem cells, and/or stem cell derivatives augment SMN gene expression in a subject, comprising administering to a subject receiving stem cells an effective amount of a sodium-proton exchanger inhibitor to increase the expression level of SMN exon 7 in a cell of the subject.

Certain embodiments herein are directed to methods wherein said sodium-proton exchanger inhibitor is 5-(N-ethyl-N-isopropyl)-amiloride.

Certain embodiments herein are directed to methods wherein said ratio of SMN transcripts having exon 7 to those lacking exon 7 is increased by at least 50%.

Certain embodiments herein are directed to methods wherein stem cells and/or derivatives are administered together with an effective amount of composition comprising a sodium-proton exchanger inhibitor and a pharmaceutically acceptable carrier or salt, to a subject with spinal muscular atrophy to ameliorate a symptom of spinal muscular atrophy.

Certain embodiments herein are directed to methods wherein the sodium-proton exchanger inhibitor is 5-(N-ethyl-N-isopropyl)-amiloride.

Certain embodiments herein are directed to methods wherein the composition is further administered in combination with an additional agent comprising histone deacetylase inhibitor, hydroxyurea, anthracycline antibiotic, phosphatase inhibitor, nonsteroidal anti-inflammatory drug, cyclooxygenase inhibitor, tobramycin, amikacin, ribonucleotide reductase inhibitor, or cell cycle inhibitor.

Certain embodiments herein are directed to methods wherein the histone deacetylase inhibitor is a butyrate, valproic acid, M344, SAHA, trapoxin, or trichostatin A.

Certain embodiments herein are directed to methods wherein said exosome possesses a lipid raft.

Certain embodiments herein are directed to methods wherein said exosome expresses antigenic markers on surface of said exosome, wherein said antigenic markers are selected from a group comprising of: a) CD9; b) CD63; c) CD81; d) ANXA2; e) ENO1; f) HSP9OAA1; g) EEF1A1; h) YWHAE; i) SDCBP; j) PDCD6IP; k) ALB; 1) YWHAZ; m) EEF2; n) ACTG1; o) LDHA; p) HSP90AB1; q) ALDOA; r) MSN; s) ANXA5; t) PGK1; and u) CFL1.

Certain embodiments herein are directed to methods wherein said mesenchymal stem cells are transfected to over express genes defective in spinal muscular atrophy.

Certain embodiments herein are directed to methods wherein said genes defective in spinal muscular atrophy are survival of motor neuron 1 (SMN1) gene and SMN2 gene.

### DESCRIPTION OF THE INVENTION

For the practice of the invention, a preferred embodiment is the administration of mesenchymal stem cells (MSC) at concentrations and frequencies sufficient to prevent, inhibit progression of or reverse SMA. Without being bound to theory, administration of said mesenchymal stem cells may be in the form of cells themselves, extracts of the cells, lysates, or nucleic acid compositions, said administration, while possessing ability to reduce and/or reverse pathology of SMA, may function through means including restoration of mitochondrial enzymes, protection of neural cells from cellular death, stimulation of neural regeneration, and/or providing transfer of genetic material. The invention teaches the use of mesenchymal stem cells from allogeneic sources for treatment of spinal muscular atrophy. It is known that at a cellular level, patients with SMA possess a swollen and chromatolysis-type morphology in motor neurons, suggesting some degree of underlying apoptosis [26]. Accordingly, in one embodiment of the invention, disclosed are methods of inhibiting apoptosis of cells from SMA patients by administration of stem cells or products thereof. Without being bound to theory, other mechanisms of mesenchymal stem cell inhibition of SMA progression and/or reversion of pathology may be achieved through suppression of skeletal muscle apoptosis and/or regeneration. It is known that patients with SMA suffer from skeletal muscle apoptosis [27, 28], although it is not clear whether the apoptosis is caused directly by an inherent defect in the muscle cell, the lack of proper communication with the upstream motor neuron, or a combination of both.

“Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage [29, 30]. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or mesenchymal stem cell can be used interchangeably. Said MSC can be derived from any tissue including, but not limited to, bone marrow [31-35], adipose tissue [36, 37], amniotic fluid [38, 39], endometrium [40-43], trophoblast-associated tissues [44], human villous trophoblasts [45], cord blood [46], Wharton jelly [47-49], umbilical cord tissue [50], placenta [51], amniotic tissue [52-54], derived from pluripotent stem cells [55-59], and tooth.

In some definitions of “MSC”, said cells include cells that are CD34 positive upon initial isolation from tissue but are similar to cells described about phenotypically and functionally. As used herein, “MSC” may include cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion.

Furthermore, as used herein, in some contexts, “MSC” includes cells described in the literature as bone marrow stromal stem cells (BMSSC) [60], marrow-isolated adult multipotent inducible cells (MIAMI) cells [61, 62], multipotent adult progenitor cells (MAPC) [63-66], MultiStem®, Prochymal [67-71], remestemcel-L [72], Mesenchymal Precursor Cells (MPCs) [73], Dental Pulp Stem Cells (DPSCs) [74], PLX cells [75], Ixmyelocel-T [76], NurOwn™ [77], Stemedyne™-MSC, Stempeucel® [78, 79], HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs) [80].

In other embodiments of the invention, stem cells, and/or products thereof are utilized for gene correction/replacement. One gene whose expression is desired, which is known to be lacking in SMA is the neuronal apoptosis inhibitory protein (NAIP) [26]. In one embodiment mesenchymal stem cells are administered from healthy donors into patients with SMA. Specific types of mesenchymal stem cells include umbilical cord tissue mesenchymal stem cells, bone marrow mesenchymal stem cells, and/or adipose derived mesenchymal stem cells. In some embodiments stem cells are screened for expression of high expression of NAIP. It is known that NAIP is a member of the inhibitor of apoptosis (IAP) family of proteins which were first identified in baculoviruses [81], and subsequently in mammals [82]. The IAP family members are potent suppressors of apoptosis in a wide variety of cultured cell lines irrespective of tissue of origin [83, 84]. NAIP possesses several mechanisms of blocking apoptosis, a major one being directly binding and inactivation of caspases 3 and 7 [85]. Interestingly, the NAIP gene is found in the SMA region of chromosome 5q13.1 (same region where SMN1 resides) and is deleted in <50% of SMA-I patients but only in 10-20% of type II and III patients, suggesting a contribution to disease progression [26]. Confirmation of clinical significance of NAIP deletion has been performed by subsequent studies. An investigation of 232 Chinese SMA patients revealed that NAIP copy numbers correlated positively with the median onset age, and that risk of death is higher for patients with fewer copies of NAIP [86], a previous study in a similar population also found negative correlation between NAIP and progression of SMA [87]. These studies were confirmed in various populations including Italians [88], Tunisians [89, 90], Spanish [91], and Americans [92].

It is the goal of the current invention to utilized mesenchymal stem cells as gene replacement means in order to replicate Interventional studies in neurodegenerative conditions which have demonstrated that forced NAIP expression by means of adenoviral transfection leads to reduction in neuronal death, including in ischemic models [93, 94]. In the scope of the current invention mesenchymal stem cells are capable of transferring therapeutic levels of NAIP to target cells that are deficient. Without being bound to mechanism, mesenchymal stem cells provide anti-inflammatory activities that further suppress progression of SMA. In another embodiment of the invention, mesenchymal stem cells provide functional SMN protein, and/or SMA gene, and/or SMN RNA transcripts to cells that are deficient. It is known that the SMN gene deletion/mutations are found SMA patients [95, 96]. In humans there are two versions of the SMN gene: SMN-1, which is telomeric gene and was found to be either lacking or interrupted in 226 of 229 patients, and patients retaining this gene (3 of 229) carry either a point mutation (Y272C) or short deletions in the consensus splice sites of introns 6 and 7. The SMN II gene is centromeric and acts to protect against loss of SMN-I. Unfortunately, the SMN-II protein product is defective, with rapid degradation, due to skipping of exon-7. Patients who have more copies of SMN-II genes have less severe forms of SMA. For example, SMA-I patients usually have 1-2 copies of SMN-II, whereas patients with SMA-2 have 3 copies, SMA-III have 3 copies and SMA-IV have >4 copies. The biological relevance of SMN to SMA has been conclusively proven in that mice lacking SMN-I are embryonically lethal, whereas when these knockout mice are made to express SMN-II a pathology similar to human SMA develops.

In accordance with the invention presented herein, the words “cell culture” and “culturing of cells” refer to the maintenance and propagation of cells and preferably human, human-derived and animal cells in vitro.

In accordance with the invention presented herein, the words “Cell culture medium” is used for the maintenance of cells in culture in vitro. For some cell types, the medium may also be sufficient to support the proliferation of the cells in culture. A medium according to the present invention provides nutrients such as energy sources, amino acids and anorganic ions. Additionally, it may contain a dye like phenol red, sodium pyruvate, several vitamins, free fatty acids, antibiotics, anti-oxidants and trace elements. For culturing the mesenchymal stem cells that are dedifferentiated into stem cells, or stem cell-like cells according to the present invention any standard medium such as Iscove's Modified Dulbecco's Media (IMDM), alpha-MEM, Dulbecco's Modified Eagle Media (DMEM), RPMI Media and McCoy's Medium is suitable before reprogramming. Ones the cells have been reprogrammed, they can in a preferred embodiment be cultured in embryonic stem cell medium.

In accordance with the invention presented herein, the word “Transfection” refers to a method of gene delivery that introduces a foreign nucleotide sequences (e.g. DNA/RNA or protein molecules) into a cell preferably by a viral or non-viral method. In preferred embodiments according to the present invention foreign DNA/RNA/proteins are introduced to a cell by transient transfection of an expression vector encoding a polypeptide of interest, whereby the foreign DNA/RNA/proteins is introduced but eliminated over time by the cell and during mitosis. By “transient transfection” is meant a method where the introduced expression vectors and the polypeptide encoded by the vector, are not permanently integrated into the genome of the host cell, or anywhere in the cell, and therefore may be eliminated from the host cell or its progeny over time. Proteins, polypeptides, or other compounds can also be delivered into a cell using transfection methods.

In accordance with the invention presented herein, the concept of identifying the “sufficient period of time” to allow stable expression of the at least one gene regulator in absence of the reprogramming agent and the “sufficient period of time” in which the cell is to be maintained in culture conditions supporting the transformation of the desired cell is within the skill of those in the art. The sufficient or proper time period will vary according to various factors, including but not limited to, the particular type and epigenetic status of cells (e.g. the cell of the first type and the desired cell), the amount of starting material (e.g. the number of cells to be transformed), the amount and type of reprogramming agent(s), the gene regulator(s), the culture conditions, presence of compounds that speed up reprogramming (ex, compounds that increase cell cycle turnover, modify the epigenetic status, and/or enhance cell viability), etc. In various embodiments the sufficient period of time to allow a stable expression of the at least one gene regulator in absence of the reprogramming agent is about 1 day, about 2-4 days, about 4-7 days, about 1-2 weeks, about 2-3 weeks or about 3-4 weeks. In various embodiments the sufficient period of time in which the cells are to be maintained in culture conditions supporting the transformation of the desired cell and allow a stable expression of a plurality of secondary genes is about 1 day, about 2-4 days, about 4-7 days, or about 1-2 weeks, about 2-3 weeks, about 3-4 weeks, about 4-6 weeks or about 6-8 weeks. In preferred embodiments, at the end of the transformation period, the number of transformed desired cells is substantially equivalent or even higher than an amount of cells a first type provided at the beginning.

Said MSC may be expanded and utilized by administration themselves, or may be cultured in a growth media in order to obtain conditioned media, the term Growth Medium generally refers to a medium sufficient for the culturing of umbilicus-derived cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium.

Also relating to the present invention, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like.

Mesenchymal stem cells (“MSC”) may be derived from the embryonal mesoderm and subsequently have been isolated from adult bone marrow and other adult tissues. They can be differentiated to form muscle, bone, cartilage, fat, marrow stroma, and tendon. Mesoderm also differentiates into visceral mesoderm which can give rise to cardiac muscle, smooth muscle, or blood islands consisting of endothelium and hematopoietic progenitor cells. The differentiation potential of the mesenchymal stem cells that have been described thus far is limited to cells of mesenchymal origin, including the best characterized mesenchymal stem cell (See Pittenger, et al. Science (1999) 284: 143-147 and U.S. Pat. No. 5,827,740 (SH2.sup.+SH4.sup.+CD29.sup.+CD44.sup.+CD71.sup.+CD90.sup.+CD106.sup.+CD120a.sup.+CD124.sup.+CD14.sup.−CD34.sup.−CD45.sup.−)). The invention teaches the use of various mesenchymal stem cells as a means of treating SMA. In some embodiments, mesenchymal stem cell therapy is administered together with drugs used experimentally for SMA in order to augment efficacy. For example, it is known phenylbutyrate is an experimental drug for treatment of SMA. Phenylbutyrate, is a drug that approved by the FDA to treat urea cycle disorders because its metabolites offer an alternative pathway to the urea cycle to allow excretion of excess nitrogen. Interestingly, phenylbutyrate also has been shown to act as a histone deacetylase (HDAC) inhibitor by increasing the acetylation of histones, thereby releasing constraints on the DNA template and reactivating genes that are epigenetically inhibited [97, 98]. In mice and man treatment with phenylbutyrate leads to re-expression of fetal hemoglobin [99, 100]. By screening for drugs capable of increasing SMN II production, Chang et al identified that sodium butyrate was capable of rescuing cell lines in vitro. Specifically, they showed the compound increased the amount of exon 7-containing SMN protein by changing the alternative splicing pattern of exon 7 in the SMN2 gene. Using an SMA-like mouse model it was shown that sodium butyrate administration caused increased expression of SMN protein in motor neurons of the spinal cord, which was associated with improvement of SMA clinical symptoms [101]. Similar SMN protein restoration by inclusion of the exon-7 from the SNM2 gene was reported by treatment of SMA patient biopsy derived fibroblasts with 4-phenybutyrate. Investigators treated fibroblast cell cultures from 16 SMA patients affected by different clinical severities with 4-phenylbutyrate, and full-length SMN2 transcripts were measured by real-time PCR. In all cell cultures, except one, treatment caused an increase in full-length SMN2 transcripts, ranging from 50 to 160% in SMA-I and from 80 to 400% cultures from SMA-II and SMA-III patients. The generated protein appeared to be functional in the cell lines since treatment increased the number of gems that possessed expression of SMN protein [102]. The first trial of phenylbutyrate comprised of 10 patients afflicted with SMA-II who were administered 500 mg/kg per day. The authors reported a significant increase in the scores of the Hammersmith functional scale between the baseline and both 3-weeks and 9-weeks assessments [24]. Analysis of leukocytes from treated patients in this trial revealed augmented SMN levels after phenylbutyrate treatment [103]. A double blind, placebo controlled trial utilizing a similar dosing regimen was conducted in 107 children, unfortunately, no functional improvement over placebo was observed [20]. Accordingly, in one embodiment of the invention, mesenchymal stem cells are administered together with phenylbutyrate using known regimens that are incorporated by reference [104-107]. For the purposes of the invention phenylbutyrate includes sodium phenylbutyrate, 4-phenylbutyrate, glycerol phenylbutyrate, as well as sodium butyrate, glycerol butyrate, and[108] prodrugs-glyceryl tributyrate (BA3G) and VX563. For the purpose of the invention, another HDAC inhibitor, valproic acid, also demonstrated ability to increase SMN protein by overcoming defect of exon-7 skipping in the SMN-2 gene. Clinical studies have reported pharmacological doses of valproic acid, which is utilized as an anti-epileptic clinically, to treat fibroblast cultures derived from SMA patients. It was shown that level of full-length SMN2 mRNA/protein increased 2- to 4-fold in response to in vitro treatment [109]. These results were reproduced by independent groups, who also demonstrated that manipulation of HDACs increases SMN, in part through upregulating the SR-like splicing factor Htra2-beta 1, which is involved in prevention of the exon skipping [110, 111]. Furthermore, another study was conducted in a 7 patient trial of valproate in adult patients with SMA type III/IV, who were treated for an average of 8 months. The treated patients had objective increases quantitative muscle strength and subjective function [22]. Similar data was generated in another 6 patient pilot trial with a similar concentration and frequency of administration of valproate [23]. However, larger double-blind, placebo controlled trials did not show any benefit [112]. Accordingly, in one embodiment of the invention, enhancement of HDAC efficacy at a clinical level by administration of stem cells is conceived.

In one embodiment MSC donor lots are generated from umbilical cord tissue. Means of generating umbilical cord tissue MSC have been previously published and are incorporated by reference [46, 49, 113-117]. The term “umbilical tissue derived cells (UTC)” refers, for example, to cells as described in U.S. Pat. No. 7,510,873, U.S. Pat. No. 7,413,734, U.S. Pat. No. 7,524,489, and U.S. Pat. No. 7,560,276. The UTC can be of any mammalian origin e.g. human, rat, primate, porcine and the like. In one embodiment of the invention, the UTC are derived from human umbilicus. umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle). In addition, these isolated human umbilicus-derived cells express a gene for each of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes.

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes.

Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In one embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activites selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIB ERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest. While the use of enzyme is presently preferred, it is not required for isolation methods as provided herein.

Methods based on mechanical separation alone may be successful in isolating the instant cells from the umbilicus as discussed above. The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells. Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 200 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR,DP, DQ.

One of skill in the art will understand that there exist numerous alternative steps for facilitating cell reprogramming which may be applied to mesenchymal stem cells. These methods include the destabilizing the cell's cytoskeletal structure (for example, by exposing the cell to cytochalasin B), loosening the chromatin structure of the cell (for example, by using agents such as 5 azacytidine (5-Aza) and Valproic acid (VPA) or DNA demethylator agents such as MBD2), transfecting the cell with one or more expression vector(s) containing at least one cDNA encoding a dedifferentiating factor(for example, OCT4, SOX-2, NANOG, or KLF), using an appropriate medium for the desired cell of a different type and an appropriate differentiation medium to induce dedifferentiation of the mesenchymal stem cells, inhibiting repressive pathways that negatively affects induction into commitment the desired cell of a different type, growing the cells on an appropriate substrate for the desired cell of a different type, and growing the cells in an environment that the desired cell of a different type (or “-like” cell) would be normally exposed to in vivo such as the proper temperature, pH and low oxygen environment (for example about 2-5% O.sub.2). In various embodiments, the invention encompasses these and other related methods and techniques for facilitating cell reprogramming/dedifferentiation.

Treatment of SMA may require various combinatorial approaches within the practice of the current invention. Specifically, administration of stem cell derived factors, including lysates, conditioned media, microvesicles, apoptotic bodies, mitochondria or exosomes. In one embodiment of the invention, exosomes are purified from mesenchymal stem cells by obtaining a mesenchymal stem cell conditioned medium, concentrating the mesenchymal stem cell conditioned medium, subjecting the concentrated mesenchymal stem cell conditioned medium to size exclusion chromatography, selecting UV absorbent fractions at 220 nm, and concentrating fractions containing exosomes.

Exosomes, also referred to as “particles” may comprise vesicles or a flattened sphere limited by a lipid bilayer. The particles may comprise diameters of 40-100 nm. The particles may be formed by inward budding of the endosomal membrane. The particles may have a density of .about.1.13-1.19 g/ml and may float on sucrose gradients. The particles may be enriched in cholesterol and sphingomyelin, and lipid raft markers such as GM1, GM3, flotillin and the src protein kinase Lyn. The particles may comprise one or more proteins present in mesenchymal stem cells or mesenchymal stem cell conditioned medium (MSC-CM), such as a protein characteristic or specific to the MSC or MSC-CM. They may comprise RNA, for example miRNA. Said particles may possess one or more genes or gene products found in MSCs or medium which is conditioned by culture of MSCs. The particle may comprise molecules secreted by the MSC. Such a particle, and combinations of any of the molecules comprised therein, including in particular proteins or polypeptides, may be used to supplement the activity of, or in place of, the MSCs or medium conditioned by the MSCs for the purpose of for example treating or preventing a disease. Said particle may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. In particular, the particle may comprise one or more tetraspanins. The particles may comprise mRNA and/or microRNA. The particle may be used for any of the therapeutic purposes that the MSC or MSC-CM may be put to use.

In one embodiment, MSC exosomes, or particles may be produced by culturing mesenchymal stem cells in a medium to condition it. The mesenchymal stem cells may comprise human umbilical tissue derived cells which possess markers selected from a group comprising of CD90, CD73 and CD105. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or b-mercaptoethanol, or any combination thereof. The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrane. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The r.sub.h of particles in this peak is about 45-55 nm. Such fractions comprise mesenchymal stem cell particles such as exosomes.

In some embodiments of the invention, treatment of SMA is performed by administration of cellular lysate from regenerative cells. Said regenerative cells may be mesenchymal stem cells, in one preferred embodiment said mesenchymal stem cells are derived from the umbilical cord. Derivation of mesenchymal stem cells from umbilical cord/Wharton's Jelly for clinical applications are described in the art and incorporated by reference [118-126]. For practice of the invention, xenogeneic free media may be used to grow mesenchymal stem cells to reduce possibility of sensitization from components such as fetal calf serum [50, 127-133]. In some embodiments of the invention, mesenchymal stem cells are pretreated using ways of enhancing regenerative activity, said means include treatment with histone deacetylase inhibitors such as valproic acid, GSK-3 inhibitors such as lithium [134-139], culture under hypoxia, and treatment with carbon monoxide [140].

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA , washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody . Confluent MSC in 175 cm² flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington DC, USA). MSCs harvested from each 10-flask set were stored for up to 4 h at 4° C. and combined at the end of the harvest. A total of 2-10′10⁶ MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′10⁷ cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1′10⁶ cells per ml in 175 cm² tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′10⁶ per 175 cm². Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram. Treatements herein can be delivered using any suitable schedule, including at least once a year, or preferably 2 or more times a year for continuous treatment.

In one embodiment, hematopoietic stem cells are CD34+ cells isolated from the peripheral blood, bone marrow, or umbilical cord blood. Specifically, the hematopoietic stem cells may be derived from the blood system of mammalian animals, include but not limited to human, mouse, rat, and these hematopoietic stem cells may be harvested by isolating from the blood or tissue organs in mammalian animals. Hematopoietic stem cells may be harvested from a donor by any known methods in the art. For example, U.S. Pub. 2013/0149286 details procedures for obtaining and purifying stem cells from mammalian cadavers. Stem cells may be harvested from a human by bone marrow harvest or peripheral blood stem cell harvest, both of which are well known techniques in the art. After stem cells have been obtained from the source, such as from certain tissues of the donor, they may be cultured using stem cell expansion techniques. Stem cell expansion techniques are disclosed in U.S. Pat. No. 6,326,198 to Emerson et al., entitled “Methods and compositions for the ex vivo replication of stem cells, for the optimization of hematopoietic progenitor cell cultures, and for increasing the metabolism, GM-CSF secretion and/or IL-6 secretion of human stromal cells,” issued Dec. 4, 2001; U.S. Pat. No. 6,338,942 to Kraus et al., entitled “Selective expansion of target cell populations,” issued Jan. 15, 2002; and U.S. Pat. No. 6,335,195 to Rodgers et al., entitled “Method for promoting hematopoietic and cell proliferation and differentiation,” issued Jan. 1, 2002, which are hereby incorporated by reference in their entireties. In some embodiments, stem cells obtained from the donor are cultured in order to expand the population of stem cells. In other preferred embodiments, stem cells collected from donor sources are not expanded using such techniques. Standard methods can be used to cyropreserve the stem cells.

In some embodiments of the invention, where there are risks associated with particular types of stem cells, for example, pluripotent stem cells, said stem cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval. A wide variety of materials may be used in various embodiments for microencapsulation of stem cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers. Techniques for microencapsulation of cells that may be used for administration of stem cells are known to those of skill in the art and are described, for example, in Chang, P., et al., 1999; Matthew, H. W., et al., 1991; Yanagi, K., et al., 1989; Cal Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of stem cells. Certain embodiments incorporate stem cells into a polymer, such as a biopolymer or synthetic polymer. Examples of biopolymers include, but are not limited to, fibronectin, fibin, fibrinogen, thrombin, collagen, and proteoglycans. Other factors, such as the cytokines discussed above, can also be incorporated into the polymer. In other embodiments of the invention, stem cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

In some embodiments of the invention, mesenchymal stem cells are cultured with substances capable of maintaining said mesenchymal stem cells in an immature state, and/or maintaining high expression of genes/mitochondria necessary to prevent, inhibit, and/or reverse SMA. Said substances are selected from the group consisting of reversin, cord blood serum, lithium, a GSK-3 inhibitor, resveratrol, pterostilbene, selenium, a selenium-containing compound, EGCG ((−)-epigallocatechin-3-gallate), valproic acid and salts of valproic acid, in particular sodium valproate. In one embodiment of the present invention, a concentration of reversin from 0.5 to 10 .mu.M, preferably of 1 .mu.M is added to the mesenchymal stem cell culture. In a furthermore preferred embodiment the present invention foresees to use resveratrol in a concentration of 10 to 100 .mu.M, preferably 50 .mu.M. In a furthermore preferred embodiment the present invention foresees to use selenium or a selenium containing compound in a concentration from 0.05 to 0.5 .mu.M, preferably of 0.1 .mu.M. In another embodiment, cord blood serum is added at a concentration of 0.1%-20% volume to the volume of tissue culture media. In furthermore preferred embodiment the present invention foresees to use EGCG in a concentration from 0.001 to 0.1 .mu.M, preferably of 0.01 .mu.M. In a furthermore preferred embodiment the present invention foresees to use valproic acid or sodium valproate in a concentration from 1 to 10 .mu.M, in particular of 5 .mu.M. In some embodiments, mesenchymal stem cells are retrodifferentiated to possess higher expression of regenerative genes. Said retrodifferentiation may be achieved by cytoplasmic transfer, transfection of cytoplasm, or cell fusion with a stem cell possessing a higher level of immaturity, said stem cells including pluripotent stem cells. In such culture/coculture procedures, the cell culture medium comprises, optionally in combination with one or more of the substances specified above, at least one transient proteolysis inhibitor. The use of at least one proteolysis inhibitor in the cell culture medium of the present invention increases the time the reprogramming proteins derived from the mRNA or any endogenous genes will be present in the cells and thus facilitates in an even more improved way the reprogramming by the transfected mRNA derived factors. The present invention uses in a particular embodiment a transient proteolysis inhibitor a protease inhibitor, a proteasome inhibitor and/or a lysosome inhibitor. In an embodiment the proteosome inhibitor is selected from the group consisting of MG132, TMC-95A, TS-341 and MG262. In a furthermore preferred embodiment the protease inhibitor is selected from the group consisting of aprotinin, G-64 and leupeptine-hemisulfat. In a furthermore preferred embodiment the lysosomal inhibitor is ammonium chloride. In one embodiment the present invention also foresees a cell culture medium comprising at least one transient inhibitor of mRNA degradation. The use of a transient inhibitor of mRNA degradation increases the half-life of the reprogramming factors as well. Another embodiment of the present invention a condition suitable to allow translation of the transfected reprogramming mRNA molecules in the cells is an oxygen content in the cell culture medium from 0.5 to 21%. More particular, and without wishing to be bound to the theory, oxygen is used to further induce or increase Oct4 by triggering Oct4 via Hif1a, in these situations concentrations of oxygen lower than atmospheric concentration are used, and can be ranging from 0.1% to 10%. In a preferred embodiment conditions that are suitable to support reprogramming of the cells by the mRNA molecules in the cells are selected; more particularly, these conditions require a temperature from 30 to 38.degree. C., preferably from 31 to 37.degree. C., most preferably from 32 to 36.degree. C. The glucose content of the medium is in a preferred embodiment of the present invention below 4.6 g/l, preferably below 4.5 g/l, more preferably below 4 g/l, even more preferably below 3 g/l, particularly preferably below 2 g/I and most preferably it is 1 g/l. DMEM media containing 1 g/l glucose being preferred for the present invention are commercially available as “DMEM low glucose” from companies such as PAA, Omega Scientific, Perbio and Biosera. More particular, and without wishing to be bound to the theory, high glucose conditions adversely support aging of cells (methylation, epigenetics) in vitro which may render the reprogramming difficult. In a furthermore preferred embodiment of the present invention the cell culture medium contains glucose in a concentration from 0.1 g/l to 4.6 g/l, preferably from 0.5 g/l to 4.5 g/l and most preferably from 1 g/l to 4 g/l.

For practice of the invention, inhibitors of acetylation are used in culture of mesenchymal stem cells. This term refers to an agent that prevents the removal of the acetyl groups from the lysine residues of histones that would otherwise lead to the formation of a condensed and transcriptionally silenced chromatin. Histone deacetylase inhibitors fall into several groups, including: (1) hydroxamic acids such as trichostatin (A) [141-144], (2) cyclic tetrapeptides, (3) benzamides, (4) electrophilic ketones, and (5) aliphatic acid group of compounds such as phenylbutyrate and valporic acid. Suitable agents to inhibit histone deacetylation include, but are not limited to, valporic acid (VPA) [145-156], phenylbutyrate and Trichostatin A (TSA). One example, in the area of mesenchymal stem cells, of valproic acid enhancing pluripotency and therapeutic properties is provided by Killer et al. who showed that culture of cells with valproic acid enhanced immune regulatory and metabolic properties of mesenchymal stem cells. The culture systems described, as well as means of assessment, are provided to allow one of skill in the art to have a starting point for the practice of the current invention [157, 158]. Without being bound to theory, valproic acid in the context of the current invention may be useful to increasing in vitro proliferation of dedifferentiated mesenchymal stem cells while preventing senescence associated stress. For example, Zhai et al showed that in an in vitro pre-mature senescence model, valproic acid treatment increased cell proliferation and inhibited apoptosis through the suppression of the p16/p21 pathway. In addition, valproic acid also inhibited the G2/M phase blockage derived from the senescence stress [159].

In some embodiments of the invention, small RNAs that act as small activating RNA (saRNA) which induce activation of OCT4 expression are applied to mesenchymal stem cell to induce dedifferentiation. In some cases this is combined with histone deacetylase inhibitors and/or GSK3 inhibitors and/or DNA methyltransferase inhibitors, in order to induce a dedifferentiated phenotype in the mesenchymal stem cells. Such mesenchymal stem cells that have been dedifferentiated can subsequently be used as a source of cells for differentiation into therapeutic cells. Small RNAs that act as small activating RNAs of the OCT4 promotor are described in the following publications [160-165].

In some embodiments, mesenchymal stem cells are transfected with miRNA and dedifferentiated before differentiating into cells of relevance to SMA. Mesenchymal stem cells may be purchased from companies such as Lonza, and cultured in DMEM medium (Invitrogen, Life Technologies Ltd) containing 10% fetal bovine serum (PAA), 2 mM L-glutamine (Invitrogen, Life Technologies Ltd), 1×MEM non-essential amino acid solution, 1×Penicillin/Streptomycin (PAA) and β-mercaptoethanol (Sigma-Aldrich). Mesenchymal stem cells may be transduced using lentiviral particles containing hsa-miR-145-5p inhibitor (Genecopoeia) at MOI=40 in the presence of 5 μg/ml Polybrene (Sigma-Aldrich). Transduced cells were selected for Hygromycin resistance (50-75 μg/ml). For transient miR-145 inhibition, 1×105 mesenchymal stem cells are transfected with 100 pmoles miR-145 mirVana® miRNA inhibitor (Life Technologies Ltd) using Neon transfection system (Invitrogen). Transfection is carried out by two 1600 V pulses for 20 ms. For reprogramming, cells are transduced using CytoTune®-iPS Sendai Reprogramming Kit (Product number A1378001) (Life Technologies Ltd) according to manufacturer's instructions. The efficiency of mesenchymal stem cell dedifferentiation can be assessed by alkaline phosphatase (AP) activity staining using Alkaline Phosphatase Blue Substrate (Sigma-Aldrich) and by TRA-1-60 expression, as determined indirect immunofluorescence. Cells are washed with PBS, fixed by 4% paraformaldehyde for 10 minutes at room temperature, washed again with PBS, and incubated overnight at 4° C. with primary antibody against TRA-1-60 (MAB4360, Merck Millipore). Then cells are washed three times with PBS and incubated with Alexa 488-conjugated secondary antibody and observed under fluorescent microscope [166].

In some embodiments, mesenchymal stem cells may be synchronized in G2 by incubating the cells in the presence of aphidicolin to arrest them in S phase and then washing the cells three times by repeated centrifugation and resuspension in phosphate buffered saline (PBS), as described herein. The cells are then incubated for a length of time sufficient for cells to enter G2 phase. For example, cells with a doubling time of approximately 24 hours, may be incubated for between 6 and 12 hours to allow them to enter G2 phase. For cells with shorter or longer doubling times, the incubation time may be adjusted accordingly. In some embodiments of the invention, mesenchymal stem cells may be synchronized in mitosis by incubating them in 0.5 .mu.g/ml nocodazole for 17-20 hours, and the mitotic cells are detached by vigorous shaking. The detached G1 phase doublets may be discarded, or they may be allowed to remain with the mitotic cells which constitute the majority (over 80%) of the detached cells. The harvested detached cells are centrifuged at 500 g for 10 minutes in a 10 ml conical tube at 4.degree. C. Synchronized or unsynchronized cells may be harvested using standard methods and washed by centrifugation at 500 g for 10 minutes in a 10 ml conical tube at 4.degree. C. The supernatant is discarded, and the cell pellet is resuspended in a total volume of 50 ml of cold PBS. The cells are centrifuged at 500 g for 10 minutes at 4.degree. C. This washing step is repeated, and the cell pellet is resuspended in approximately 20 volumes of ice-cold interphase cell lysis buffer (20 mM Hepes, pH 8.2, 5 mM MgCl.sub.2, 1 mM DTT, 10 pM aprotinin, 10 pM leupeptin, 10 pM pepstatin A, 10 pM soybean trypsin inhibitor, 100 pM PMSF, and optionally 20 pg/ml cytochalasin B). The cells are sedimented by centrifugation at 800 g for 10 minutes at 4.degree. C. The supernatant is discarded, and the cell pellet is carefully resuspended in no more than one volume of interphase cell lysis buffer. The cells are incubated on ice for one hour to allow swelling of the cells. The cells are then lysed by either sonication using a tip sonicator or Dounce homogenization using a glass mortar and pestle. Cell lysis is performed until at least 90% of the cells and nuclei are lysed, which may be assessed using phase contrast microscopy. Duration and power of sonication required to lyse at least 90% of the cells and nuclei may vary depending on the type of cell used to prepare the extract.

In some embodiments, the cell lysate is placed in a 1.5-ml centrifuge tube and centrifuged at 10,000 to 15,000 g for 15 minutes at 4.degree. C. using a table top centrifuge. The tubes are removed from the centrifuge and immediately placed on ice. The supernatant is carefully collected using a 200 .mu.1 pipette tip, and the supernatant from several tubes is pooled and placed on ice. This supernatant is the cytoplasmic extract. This cell extract may be aliquoted into 20 pl volumes of extract per tube on ice and immediately flash-frozen on liquid nitrogen and stored at 80.degree. C. until use. Alternatively, the cell extract is placed in an ultracentrifuge tube on ice (e. g., fitted for an SW55 Ti rotor; Beckman). If necessary, the tube is overlayed with mineral oil to the top. The extract is centrifuged at 200,000 g for three hours at 4.degree. C. to sediment membrane vesicles contained in the cytoplasmic extract. At the end of centrifugation, the oil is discarded. The supernatant is carefully collected, pooled if necessary, and placed in a cold 1.5 ml tube on ice.

In other embodiments, mesenchymal stem cell lysate is generated by rinsing cells 3-4 times with PBS, and culture medium, such as alpha-MEM or DMEM/F12 (Gibco) is added without additives or serum. 12-24 hours later, the cells are washed twice with PBS and harvested, preferably scraped with a rubber policeman and collected in a 50 ml Falcon tube (Becton Dickinson). Then cells are washed and resuspended in ice-cold cell lysis buffer (20 mM HEPES, pH 8.2, 50 mM NaCl, 5 mM MgCl.sub.2, 1 mM dithiothreitol and a protease inhibitor cocktail), sedimented at 400 g and resuspended in one volume of cell lysis buffer. Cells are sonicated on ice in 200 .mu.1 aliquots using a sonicator fitted with a 2-mm diameter probe until all cells and nuclei are lysed, as can be judged by phase contrast microscopy. The lysate is centrifuged at 10,000-14,000 g, 15-30 minutes at 4.degree. C. to pellet the coarse material and any potentially remaining non-lysed cell. The supernatant is aliquoted, frozen and stored in liquid nitrogen or immediately used. Protein concentration of the extract is analyzed by Bradford assay, pH is adjusted to around 7.0.+−.0.4 and osmolarity is adjusted to −300 mOsm prior to use, in necessary, (by diluting with water).

In addition to cell lysate, conditioned media from cells may be utilized. Both cell lysate and conditioned media may be administered intranasally through an aerosolation means, or may be administered orally, intravenously, subcutaneously, intrarectally, intramuscularly, or sublingually.

Conditioned media may be generated in order to concentrated secreted factors, or may be utilized as a source of exosomes. In some embodiments, exosomes are concentrated by means of ultracentrifugation, chromatography, or based on adhesion to substrates. In some embodiments of the invention, mesenchymal stem cells are administered together with agents that increase expression of SMN2 protein by including the exon-7, which is usually excluded [167]. One example of such an agent is the drug Nusinersen, which is an antisense oligonucleotide designed to bind to the SMN2 pre-mRNA and promote inclusion of exon-7. The use of Nusinersen is described in the art, and examples are provided to assist in the practice of the invention. For example, in a Phase I study, 28 children, aged 2-14 years, with SMA-II and III where treated in a dose-escalating manner by intrathecal infusion. Nusinersen was well-tolerated with no safety/tolerability concerns identified. Plasma and CSF drug levels were dose-dependent, consistent with preclinical data. Extended pharmacokinetics indicated a prolonged CSF drug half-life of 4-6 months after initial clearance. A significant increase in HFMSE scores was observed at the 9-mg dose at 3 months postdose (3.1 points; p=0.016), which was further increased 9-14 months postdose (5.8 points; p=0.008) during the extension study [168]. A separate publication reported on details of intrathecal administration and safety aspects [169]. One of skill in the art will appreciate that combination protocols using Nusinersen and mesenchymal stem cells may be developed by utilization of various routes of administration, cell and drug doses.

In another embodiment the stimulation of SMN gene expression is performed by transfection in vitro of MSC and/or in vivo transfection. The use of mesenchymal stem cells together with gene addition therapy of SMA is envisioned in the practice of the invention. For practice of the invention, one is referred to previous uses of gene therapy for SMA, for example, it was reported that a single intravenous injection of self-complementary adeno-associated virus-9 carrying the human SMN cDNA (scAAV9-SMN) results in widespread transgene expression in spinal cord motor neurons in SMA mice as well as nonhuman primates and complete rescue of the disease phenotype in mice. Dosing of scAAV9-SMN delivered directly to the cerebral spinal fluid (CSF) via single injection has been shown to lead to widespread transgene expression throughout the spinal cord in mice and nonhuman primates when using a 10 times lower dose compared to the IV application. Interestingly, in nonhuman primates, lower doses than in mice can be used for similar motor neuron targeting efficiency [170].

### EXAMPLE

Female patient born on Jun. 6, 2008, to a 39-year-old mother. Patient is a result of IVF, dizygotic twin (twin B), born at 36 weeks via C-section. Mother presented with premature labor symptoms at 20 weeks requiring bed rest until delivery. Patient did not require any resuscitation after delivery and was discharged from the hospital after 5 days. At time of manuscript submission, patient was 8 years old, diagnosed with Spinal Muscular Atrophy (SMA) Type II at Age 3. Prior to diagnosis parents stated that all developmental milestones were normal; however, she demonstrated gait difficulties (increased frequency of falls) since 12 months of age (i.e., when able to walk) and lower extremity weakness, at times needing to manually move her legs, also having difficulty and using her arms to stand from a seated position and abnormal posture when seated (“W” or “T” shape).

At 28 months of age, physical exam revealed normal cognition, with lower extremity weakness, hypotonia, hyporeflexia (preserved ankle reflexes, absent patellar reflexes and hypoactive throughout) and mild swallowing difficulties, no tremors. Weakness was noted in a peripheral pattern (i.e., not localizable to brain or cord). Normal creatinine kinase, comprehensive metabolic panel and aldolase ruled out an inflammatory process or dystrophy; however genetic test performed in late January, 2011 confirmed diagnosis of SMA Type 2.

Since June of 2012, the patient has received more than 5 stem cell treatments in Panama City, Panama consisting of:

First and Second Treatment (June, 2012 and April/May, 2013): 5×10(5) umbilical cord blood CD34+NE cells+1.2×10(7) CD34+cells+2.4×10(7) Human umbilical cord-derived mesenchymal stem cells (hUC-MSCs)—delivered intravenously.

Third to Fifth Treatment (March, 2014, March, 2015, and March, 2016): 3.6×10(7) hUC-MSCs -delivered intravenously.

Prior to first treatment in Panama, parents indicated that patient presented walking difficulties, balance issues, and weakness in lower limbs. Physical examination showed a mild decrease of strength in lower limbs (4/5); upper limbs strength was 5/5 but with some limitations in fine motor skills. Reflexes were not obtainable. No tremors were noted. Patient did not fall when asked to deambulate during physical examination. During the week of treatment, the patient did not show any significant changes and/or side effects.

Post-treatment surveys completed by parents revealed that thirty days after first treatment, patient showed an almost immediate response, including improvements with her walking (more stability and falling less), balance (sitting on a small exercise ball without support) and eating (chewing and swallowing better, able to eat more solid foods). Ninety days post-treatment, the patient was reported to have further improvements in walking, with more stability, including ability to take some steps without holding the handrail. Further improvements in chewing and swallowing were also reported.

After following treatment sessions, patient continued to show almost immediate improvements in strength, balance, energy, and eating. Patient was also able to reach new milestones, such as new ability to jump, climb stairs without holding onto handrail, and run; patient was also able to take longer walks. Improvements consistently lasted approximately 8 to 9 months, after which time signs and symptoms of her condition started to return.

During each pre-treatment evaluation at Stem Cell Institute, physical exam revealed upper extremity strength was normal (5/5) and weakness in lower extremities (4/5), no tremors, deep tendon reflexes diminished in lower extremities (1+) and normal in upper limbs (2+), normal fine motor skills in upper limbs. Patient presented age-appropriate cognition. During Visit 5 pre-treatment evaluation specifically, physical exam revealed mildly abnormal gait (wide stance).

Per data collected from the Visit 5 thirty-day follow-up survey (most recent information available to date [April, 2016]), parents reported that patient has more energy (less tired), is walking better and has decreased tremors.

### REFERENCES

1. Monani, U. R., Spinal muscular atrophy: a deficiency in a ubiquitous protein; a motor neuron-specific disease. Neuron, 2005. 48(6): p. 885-96.
2. D'Amico, A., et al., Spinal muscular atrophy. Orphanet J Rare Dis, 2011. 6: p. 71.
3. Monani, U. R., et al., A single nucleotide difference that alters splicing patterns distinguishes the SMA gene SMN1 from the copy gene SMN2. Hum Mol Genet, 1999. 8(7): p. 1177-83.
4. McAndrew, P. E., et al., Identification of proximal spinal muscular atrophy carriers and patients by analysis of SMNT and SMNC gene copy number. Am J Hum Genet, 1997. 60(6): p. 1411-22.
5. Butchbach, M. E., Copy Number Variations in the Survival Motor Neuron Genes: Implications for Spinal Muscular Atrophy and Other Neurodegenerative Diseases. Front Mol Biosci, 2016. 3: p. 7.
6. Zarkov, M., et al., Association between the SMN2 gene copy number and clinical characteristics of patients with spinal muscular atrophy with homozygous deletion of exon 7 of the SMN1 gene. Vojnosanit Pregl, 2015. 72(10): p. 859-63.
7. Tiziano, F. D., et al., The Hammersmith functional score correlates with the SMN2 copy number: a multicentric study. Neuromuscul Disord, 2007. 17(5): p. 400-3.
8. Baioni, M. T. and C. R. Ambiel, Spinal muscular atrophy: diagnosis, treatment and future prospects. J Pediatr (Rio J), 2010. 86(4): p. 261-70.
9. MacLeod, M. J., et al., Prenatal onset spinal muscular atrophy. Eur J Paediatr Neurol, 1999. 3(2): p. 65-72.
10. Messina, S., et al., Feeding problems and malnutrition in spinal muscular atrophy type II. Neuromuscul Disord, 2008. 18(5): p. 389-93.
11. Kinali, M., et al., Bone mineral density in a paediatric spinal muscular atrophy population. Neuropediatrics, 2004. 35(6): p. 325-8.
12. Liu, Q. and G. Dreyfuss, A novel nuclear structure containing the survival of motor neurons protein. EMBO J, 1996. 15(14): p. 3555-65.
13. Pellizzoni, L., Chaperoning ribonucleoprotein biogenesis in health and disease. EMBO Rep, 2007. 8(4): p. 340-5.
14. Li, D. K., et al., SMN control of RNP assembly: from post-transcriptional gene regulation to motor neuron disease. Semin Cell Dev Biol, 2014. 32: p. 22-9.
15. Eggert, C., et al., Spinal muscular atrophy: the RNP connection. Trends Mol Med, 2006. 12(3): p. 113-21.
16. Borg, R. and R. J. Cauchi, GEMINs: potential therapeutic targets for spinal muscular atrophy? Front Neurosci, 2014. 8: p. 325.
17. Oprea, G. E., et al., Plastin 3 is a protective modifier of autosomal recessive spinal muscular atrophy. Science, 2008. 320(5875): p. 524-7.
18. Nicole, S., et al., Spinal muscular atrophy: recent advances and future prospects. Muscle Nerve, 2002. 26(1): p. 4-13.
19. Kissel, J. T., et al., SMA valiant trial: a prospective, double-blind, placebo-controlled trial of valproic acid in ambulatory adults with spinal muscular atrophy. Muscle Nerve, 2014. 49(2): p. 187-92.
20. Mercuri, E., et al., Randomized, double-blind, placebo-controlled trial of phenylbutyrate in spinal muscular atrophy. Neurology, 2007. 68(1): p. 51-5.
21. Chen, T. H., et al., Randomized, double-blind, placebo-controlled trial of hydroxyurea in spinal muscular atrophy. Neurology, 2010. 75(24): p. 2190-7.
22. Weihl, C. C., A. M. Connolly, and A. Pestronk, Valproate may improve strength and function in patients with type III/IV spinal muscle atrophy. Neurology, 2006. 67(3): p. 500-1.
23. Tsai, L. K., et al., Valproic acid treatment in six patients with spinal muscular atrophy. Eur J Neurol, 2007. 14(12): p. e8-9.
24. Mercuri, E., et al., Pilot trial of phenylbutyrate in spinal muscular atrophy. Neuromuscul Disord, 2004. 14(2): p. 130-5.
25. Liang, W. C., et al., The effect of hydroxyurea in spinal muscular atrophy cells and patients. J Neurol Sci, 2008. 268(1-2): p. 87-94.
26. Roy, N., et al., The gene for neuronal apoptosis inhibitory protein is partially deleted in individuals with spinal muscular atrophy. Cell, 1995. 80(1): p. 167-78.
27. Tews, D. S. and H. H. Goebel, Apoptosis-related proteins in skeletal muscle fibers of spinal muscular atrophy. J Neuropathol Exp Neurol, 1997. 56(2): p. 150-6.
28. Tews, D. S., H. H. Goebel, and H. M. Meinck, DNA-fragmentation and apoptosis-related proteins of muscle cells in motor neuron disorders. Acta Neurol Scand, 1997. 96(6): p. 380-6.
29. Martin, I., et al., A relativity concept in mesenchymal stromal cell manufacturing. Cytotherapy, 2016. 18(5): p. 613-20.
30. Uder, C., et al., Mammalian MSC from selected species: Features and applications. Cytometry A, 2017.
31. Lechanteur, C., et al., Clinical-scale expansion of mesenchymal stromal cells: a large banking experience. J Transl Med, 2016. 14(1): p. 145.
32. Yi, T., et al., Manufacture of Clinical-Grade Human Clonal Mesenchymal Stem Cell Products from Single Colony Forming Unit-Derived Colonies Based on the Subfractionation Culturing Method. Tissue Eng Part C Methods, 2015. 21(12): p. 1251-62.
33. Hanley, P. J., Therapeutic mesenchymal stromal cells: where we are headed. Methods Mol Biol, 2015. 1283: p. 1-11.
34. Nold, P., et al., Good manufacturing practice-compliant animal-free expansion of human bone marrow derived mesenchymal stroma cells in a closed hollow-fiber-based bioreactor. Biochem Biophys Res Commun, 2013. 430(1): p. 325-30.
35. Gastens, M. H., et al., Good manufacturing practice-compliant expansion of marrow-derived stem and progenitor cells for cell therapy. Cell Transplant, 2007. 16(7): p. 685-96.
36. Nicoletti, G. F., et al., Methods and procedures in adipose stem cells: state of the art and perspective for translation medicine. J Cell Physiol, 2015. 230(3): p. 489-95.
37. Siciliano, C., et al., Optimization of the isolation and expansion method of human mediastinal-adipose tissue derived mesenchymal stem cells with virally inactivated GMP-grade platelet lysate. Cytotechnology, 2015. 67(1): p. 165-74.
38. Gubar, O. S., et al., Postnatal extra-embryonic tissues as a source of multiple cell types for regenerative medicine applications. Exp Oncol, 2017. 39(3): p. 186-190.
39. Lim, R., et al., A Pilot Study Evaluating the Safety of Intravenously Administered Human Amnion Epithelial Cells for the Treatment of Hepatic Fibrosis. Front Pharmacol, 2017. 8: p. 549.
40. Zlatska, A. V., et al., Endometrial stromal cells: isolation, expansion, morphological and functional properties. Exp Oncol, 2017. 39(3): p. 197-202.
41. Lan, X., et al., Stromal Cell-Derived Factor-1 Mediates Cardiac Allograft Tolerance Induced by Human Endometrial Regenerative Cell-Based Therapy. Stem Cells Transl Med, 2017.
42. Xu, X., et al., Prolongation of Cardiac Allograft Survival by Endometrial Regenerative Cells: Focusing on B-Cell Responses. Stem Cells Transl Med, 2017. 6(3): p. 778-787.
43. Rodrigues, M. C., et al., Menstrual Blood-Derived Stem Cells: In Vitro and In Vivo Characterization of Functional Effects. Adv Exp Med Biol, 2016. 951: p. 111-121.
44. James, J. L., et al., Isolation and characterisation of a novel trophoblast side-population from first trimester placentae. Reproduction, 2015. 150(5): p. 449-62.
45. Wang, Z., et al., Transplantation of human villous trophoblasts preserves cardiac function in mice with acute myocardial infarction. J Cell Mol Med, 2017. 21(10): p. 2432-2440.
46. Schira, J., et al., Significant clinical, neuropathological and behavioural recovery from acute spinal cord trauma by transplantation of a well-defined somatic stem cell from human umbilical cord blood. Brain, 2012. 135(Pt 2): p. 431-46.
47. Ducret, M., et al., A standardized procedure to obtain mesenchymal stem/stromal cells from minimally manipulated dental pulp and Wharton's jelly samples. Bull Group Int Rech Sci Stomatol Odontol, 2016. 53(1): p. e37.
48. Van Pham, P., et al., Isolation and proliferation of umbilical cord tissue derived mesenchymal stem cells for clinical applications. Cell Tissue Bank, 2016. 17(2): p. 289-302.
49. Friedman, R., et al., Umbilical cord mesenchymal stem cells: adjuvants for human cell transplantation. Biol Blood Marrow Transplant, 2007. 13(12): p. 1477-86.
50. Emnett, R. J., et al., Evaluation of Tissue Homogenization to Support the Generation of GMP-Compliant Mesenchymal Stromal Cells from the Umbilical Cord. Stem Cells Int, 2016. 2016: p. 3274054.
51. Choi, Y. S., et al., Different characteristics of mesenchymal stem cells isolated from different layers of full term placenta. PLoS One, 2017. 12(2): p. e0172642.
52. Koike, C., et al., Characterization of amniotic stem cells. Cell Reprogram, 2014. 16(4): p. 298-305.
53. Kim, S. W., et al., Amniotic mesenchymal stem cells with robust chemotactic properties are effective in the treatment of a myocardial infarction model. Int J Cardiol, 2013. 168(2): p. 1062-9.
54. Walther, G., J. Gekas, and O. F. Bertrand, Amniotic stem cells for cellular cardiomyoplasty: promises and premises. Catheter Cardiovasc Interv, 2009. 73(7): p. 917-24.
55. Ullah, M., et al., iPS-derived MSCs from an expandable bank to deliver a prodrug-converting enzyme that limits growth and metastases of human breast cancers. Cell Death Discov, 2017. 3: p. 16064.
56. Moslem, M., et al., Kindlin-2 Modulates the Survival, Differentiation, and Migration of Induced Pluripotent Cell-Derived Mesenchymal Stromal Cells. Stem Cells Int, 2017. 2017: p. 7316354.
57. Luzzani, C. D. and S. G. Miriuka, Pluripotent Stem Cells as a Robust Source of Mesenchymal Stem Cells. Stem Cell Rev, 2017. 13(1): p. 68-78.
58. Lo Cicero, A., et al., A High Throughput Phenotypic Screening reveals compounds that counteract premature osteogenic differentiation of HGPS iPS-derived mesenchymal stem cells. Sci Rep, 2016. 6: p. 34798.
59. Sheyn, D., et al., Human Induced Pluripotent Stem Cells Differentiate Into Functional Mesenchymal Stem Cells and Repair Bone Defects. Stem Cells Transl Med, 2016. 5(11): p. 1447-1460.
60. Shi, S., et al., Bone formation by human postnatal bone marrow stromal stem cells is enhanced by telomerase expression. Nat Biotechnol, 2002. 20(6): p. 587-91.
61. Grau-Monge, C., et al., Marrow-isolated adult multilineage inducible cells embedded within a biologically-inspired construct promote recovery in a mouse model of peripheral vascular disease. Biomed Mater, 2017. 12(1): p. 015024.
62. Rahnemai-Azar, A., et al., Human marrow-isolated adult multilineage-inducible (MIAMI) cells protect against peripheral vascular ischemia in a mouse model. Cytotherapy, 2011. 13(2): p. 179-92.
63. Soeder, Y., et al., First-in-Human Case Study: Multipotent Adult Progenitor Cells for Immunomodulation After Liver Transplantation. Stem Cells Transl Med, 2015. 4(8): p. 899-904.
64. Boozer, S., et al., Global Characterization and Genomic Stability of Human MultiStem, A Multipotent Adult Progenitor Cell. J Stem Cells, 2009. 4(1): p. 17-28.
65. Maziarz, R. T., et al., Single and multiple dose MultiStem (multipotent adult progenitor cell) therapy prophylaxis of acute graft-versus-host disease in myeloablative allogeneic hematopoietic cell transplantation: a phase 1 trial. Biol Blood Marrow Transplant, 2015. 21(4): p. 720-8.
66. Plessers, J., et al., Clinical-Grade Human Multipotent Adult Progenitor Cells Block CD8+ Cytotoxic T Lymphocytes. Stem Cells Transl Med, 2016. 5(12): p. 1607-1619.
67. Kebriaei, P., et al., Adult human mesenchymal stem cells added to corticosteroid therapy for the treatment of acute graft-versus-host disease. Biol Blood Marrow Transplant, 2009. 15(7): p. 804-11.
68. Allison, M., Genzyme backs Osiris, despite Prochymal flop. Nat Biotechnol, 2009. 27(11): p. 966-7.
69. Hare, J. M., et al., A randomized, double-blind, placebo-controlled, dose-escalation study of intravenous adult human mesenchymal stem cells (prochymal) after acute myocardial infarction. J Am Coll Cardiol, 2009. 54(24): p. 2277-86.
70. Prasad, V. K., et al., Efficacy and safety of ex vivo cultured adult human mesenchymal stem cells (Prochymal) in pediatric patients with severe refractory acute graft-versus-host disease in a compassionate use study. Biol Blood Marrow Transplant, 2011. 17(4): p. 534-41.
71. Patel, A. N. and J. Genovese, Potential clinical applications of adult human mesenchymal stem cell (Prochymal(R)) therapy. Stem Cells Cloning, 2011. 4: p. 61-72.
72. Mannon, P. J., Remestemcel-L: human mesenchymal stem cells as an emerging therapy for Crohn's disease. Expert Opin Biol Ther, 2011. 11(9): p. 1249-56.
73. Wang, Y., et al., Safety, tolerability, clinical, and joint structural outcomes of a single intra-articular injection of allogeneic mesenchymal precursor cells in patients following anterior cruciate ligament reconstruction: a controlled double-blind randomised trial. Arthritis Res Ther, 2017. 19(1): p. 180.
74. Kolar, M. K., et al., The neurotrophic effects of different human dental mesenchymal stem cells. Sci Rep, 2017. 7(1): p. 12605.
75. Prather, W. R., A. Toren, and M. Meiron, Placental-derived and expanded mesenchymal stromal cells (PLX-I) to enhance the engraftment of hematopoietic stem cells derived from umbilical cord blood. Expert Opin Biol Ther, 2008. 8(8): p. 1241-50.
76. Patel, A. N., et al., Ixmyelocel-T for patients with ischaemic heart failure: a prospective randomised double-blind trial. Lancet, 2016. 387(10036): p. 2412-21.
77. Perets, N., et al., Long term beneficial effect of neurotrophic factors-secreting mesenchymal stem cells transplantation in the BTBR mouse model of autism. Behav Brain Res, 2017. 331: p. 254-260.
78. Gupta, P. K., et al., Administration of Adult Human Bone Marrow-Derived, Cultured, Pooled, Allogeneic Mesenchymal Stromal Cells in Critical Limb Ischemia Due to Buerger's Disease: Phase II Study Report Suggests Clinical Efficacy. Stem Cells Transl Med, 2017. 6(3): p. 689-699.
79. Thej, C., et al., Development of a surrogate potency assay to determine the angiogenic activity of Stempeucel(R), a pooled, ex-vivo expanded, allogeneic human bone marrow mesenchymal stromal cell product. Stem Cell Res Ther, 2017. 8(1): p. 47.
80. Zhu, M., et al., Manual isolation of adipose-derived stem cells from human lipoaspirates. J Vis Exp, 2013(79): p. e50585.
81. Clem, R. J. and C. S. Duckett, The iap genes: unique arbitrators of cell death. Trends Cell Biol, 1997. 7(9): p. 337-9.
82. Miller, L. K., An exegesis of IAPs: salvation and surprises from BIR motifs. Trends Cell Biol, 1999. 9(8): p. 323-8.
83. Deveraux, Q. L. and J. C. Reed, IAP family proteins—suppressors of apoptosis. Genes Dev, 1999. 13(3): p. 239-52.
84. LaCasse, E. C., et al., The inhibitors of apoptosis (IAPs) and their emerging role in cancer. Oncogene, 1998. 17(25): p. 3247-59.
85. Maier, J. K., et al., The neuronal apoptosis inhibitory protein is a direct inhibitor of caspases 3 and 7. J Neurosci, 2002. 22(6): p. 2035-43.
86. Qu, Y. J., et al., Association of copy numbers of survival motor neuron gene 2 and neuronal apoptosis inhibitory protein gene with the natural history in a Chinese spinal muscular atrophy cohort. J Child Neurol, 2015. 30(4): p. 429-36.
87. He, J., et al., Molecular analysis of SMN1, SMN2, NAIP, GTF2H2, and H4F5 genes in 157 Chinese patients with spinal muscular atrophy. Gene, 2013. 518(2): p. 325-9.
88. Capon, F., et al., Deletion analysis of SMN and NAIP genes in spinal muscular atrophy Italian families. Muscle Nerve, 1996. 19(3): p. 378-80.
89. Mrad, R., et al., Molecular analysis of the SMN1 and NAIP genes in 60 Tunisian spinal muscular atrophy patients. Tunis Med, 2006. 84(8): p. 465-9.
90. Rekik, I., et al., Deletion analysis of SMN and NAIP genes in Tunisian patients with spinal muscular atrophy. Ann Indian Acad Neurol, 2013. 16(1): p. 57-61.
91. Velasco, E., et al., Molecular analysis of the SMN and NAIP genes in Spanish spinal muscular atrophy (SMA) families and correlation between number of copies of cBCD541 and SMA phenotype. Hum Mol Genet, 1996. 5(2): p. 257-63.
92. Campbell, L., et al., Genomic variation and gene conversion in spinal muscular atrophy: implications for disease process and clinical phenotype. Am J Hum Genet, 1997. 61(1): p. 40-50.
93. Xu, D. G., et al., Elevation of neuronal expression of NAIP reduces ischemic damage in the rat hippocampus. Nat Med, 1997. 3(9): p. 997-1004.
94. Hara, H., [Involvement of caspase on apoptosis in ischemia-induced neuronal cell death: usefulness of caspase inhibitors for stroke therapy]. Nihon Yakurigaku Zasshi, 1999. 113(2): p. 97-111.
95. Lefebvre, S., et al., Identification and characterization of a spinal muscular atrophy-determining gene. Cell, 1995. 80(1): p. 155-65.
96. Ahmad, S., et al., Molecular Mechanisms of Neurodegeneration in Spinal Muscular Atrophy. J Exp Neurosci, 2016. 10: p. 39-49.
97. Sealy, L. and R. Chalkley, The effect of sodium butyrate on histone modification. Cell, 1978. 14(1): p. 115-21.
98. Vidali, G., et al., Butyrate suppression of histone deacetylation leads to accumulation of multiacetylated forms of histones H3 and H4 and increased DNase I sensitivity of the associated DNA sequences. Proc Natl Acad Sci U S A, 1978. 75(5): p. 2239-43.
99. Weinberg, R. S., et al., Butyrate increases the efficiency of translation of gamma-globin mRNA. Blood, 2005. 105(4): p. 1807-9.
100. Perrine, S. P., et al., A short-term trial of butyrate to stimulate fetal-globin-gene expression in the beta-globin disorders. N Engl J Med, 1993. 328(2): p. 81-6.
101. Chang, J. G., et al., Treatment of spinal muscular atrophy by sodium butyrate. Proc Natl Acad Sci U S A, 2001. 98(17): p. 9808-13.
102. Andreassi, C., et al., Phenylbutyrate increases SMN expression in vitro: relevance for treatment of spinal muscular atrophy. Eur J Hum Genet, 2004. 12(1): p. 59-65.
103. Brahe, C., et al., Phenylbutyrate increases SMN gene expression in spinal muscular atrophy patients. Eur J Hum Genet, 2005. 13(2): p. 256-9.
104. Al-Keilani, M. S. and N. A. Al-Sawalha, Potential of Phenylbutyrate as Adjuvant Chemotherapy: An Overview of Cellular and Molecular Anticancer Mechanisms. Chem Res Toxicol, 2017. 30(10): p. 1767-1777.
105. Berry, S. A., et al., Safety and efficacy of glycerol phenylbutyrate for management of urea cycle disorders in patients aged 2 months to 2 years. Mol Genet Metab, 2017.
106. De Las Heras, J., et al., An update on the use of benzoate, phenylacetate and phenylbutyrate ammonia scavengers for interrogating and modifying liver nitrogen metabolism and its implications in urea cycle disorders and liver disease. Expert Opin Drug Metab Toxicol, 2017. 13(4): p. 439-448.
107. Laemmle, A., T. Stricker, and J. Haberle, Switch from Sodium Phenylbutyrate to Glycerol Phenylbutyrate Improved Metabolic Stability in an Adolescent with Ornithine Transcarbamylase Deficiency. JIMD Rep, 2017. 31: p. 11-14.
108. Butchbach, M. E. R., et al., Protective effects of butyrate-based compounds on a mouse model for spinal muscular atrophy. Exp Neurol, 2016. 279: p. 13-26.
109. Brichta, L., et al., Valproic acid increases the SMN2 protein level: a well-known drug as a potential therapy for spinal muscular atrophy. Hum Mol Genet, 2003. 12(19): p. 2481-9.
110. Hofmann, Y., et al., Htra2-beta 1 stimulates an exonic splicing enhancer and can restore full-length SMN expression to survival motor neuron 2 (SMN2). Proc Natl Acad Sci U S A, 2000. 97(17): p. 9618-23.
111. Helmken, C. and B. Wirth, Exclusion of Htra2-beta1, an up-regulator of full-length SMN2 transcript, as a modifying gene for spinal muscular atrophy. Hum Genet, 2000. 107(6): p. 554-8.
112. Swoboda, K. J., et al., Phase II open label study of valproic acid in spinal muscular atrophy. PLoS One, 2009. 4(5): p. e5268.
113. Van Pham, P., et al., Isolation and proliferation of umbilical cord tissue derived mesenchymal stem cells for clinical applications. Cell Tissue Bank, 2015.
114. Fazzina, R., et al., A new standardized clinical-grade protocol for banking human umbilical cord tissue cells. Transfusion, 2015. 55(12): p. 2864-73.
115. Bieback, K., Platelet lysate as replacement for fetal bovine serum in mesenchymal stromal cell cultures. Transfus Med Hemother, 2013. 40(5): p. 326-35.
116. Stanko, P., et al., Comparison of human mesenchymal stem cells derived from dental pulp, bone marrow, adipose tissue, and umbilical cord tissue by gene expression. Biomed Pap Med Fac Univ Palacky Olomouc Czech Repub, 2014. 158(3): p. 373-7.
117. Hartmann, I., et al., Umbilical cord tissue-derived mesenchymal stem cells grow best under GMP-compliant culture conditions and maintain their phenotypic and functional properties. J Immunol Methods, 2010. 363(1): p. 80-9.
118. Can, A., F. T. Celikkan, and O. Cinar, Umbilical cord mesenchymal stromal cell transplantations: A systemic analysis of clinical trials. Cytotherapy, 2017.
119. Bilal, M., A. Haseeb, and M. A. Sher Khan, Intracoronary infusion of Wharton's jelly-derived mesenchymal stem cells: a novel treatment in patients of acute myocardial infarction. J Pak Med Assoc, 2015. 65(12): p. 1369.
120. Gao, L. R., et al., Intracoronary infusion of Wharton's jelly-derived mesenchymal stem cells in acute myocardial infarction: double-blind, randomized controlled trial. BMC Med, 2015. 13: p. 162.
121. Chatzistamatiou, T. K., et al., Optimizing isolation culture and freezing methods to preserve Wharton's jelly's mesenchymal stem cell (MSC) properties: an MSC banking protocol validation for the Hellenic Cord Blood Bank. Transfusion, 2014. 54(12): p. 3108-20.
122. Liu, X., et al., A preliminary evaluation of efficacy and safety of Wharton's jelly mesenchymal stem cell transplantation in patients with type 2 diabetes mellitus. Stem Cell Res Ther, 2014. 5(2): p. 57.
123. Wu, K. H., et al., Human application of ex vivo expanded umbilical cord-derived mesenchymal stem cells: enhance hematopoiesis after cord blood transplantation. Cell Transplant, 2013. 22(11): p. 2041-51.
124. Kim, D. W., et al., Wharton's jelly-derived mesenchymal stem cells: phenotypic characterization and optimizing their therapeutic potential for clinical applications. Int J Mol Sci, 2013. 14(6): p. 11692-712.
125. Batsali, A. K., et al., Mesenchymal stem cells derived from Wharton's Jelly of the umbilical cord: biological properties and emerging clinical applications. Curr Stem Cell Res Ther, 2013. 8(2): p. 144-55.
126. Hu, J., et al., Long term effects of the implantation of Wharton's jelly-derived mesenchymal stem cells from the umbilical cord for newly-onset type 1 diabetes mellitus. Endocr J, 2013. 60(3): p. 347-57.
127. de Soure, A. M., et al., Scalable microcarrier-based manufacturing of mesenchymal stem/stromal cells. J Biotechnol, 2016. 236: p. 88-109.
128. Fernandes-Platzgummer, A., et al., Clinical-Grade Manufacturing of Therapeutic Human Mesenchymal Stem/Stromal Cells in Microcarrier-Based Culture Systems. Methods Mol Biol, 2016. 1416: p. 375-88.
129. Mizukami, A., et al., Stirred tank bioreactor culture combined with serum-/xenogeneic-free culture medium enables an efficient expansion of umbilical cord-derived mesenchymal stem/stromal cells. Biotechnol J, 2016. 11(8): p. 1048-59.
130. Smith, J. R., et al., Standardizing Umbilical Cord Mesenchymal Stromal Cells for Translation to Clinical Use: Selection of GMP-Compliant Medium and a Simplified Isolation Method. Stem Cells Int, 2016. 2016: p. 6810980.
131. Carmelo, J. G., et al., Scalable ex vivo expansion of human mesenchymal stem/stromal cells in microcarrier-based stirred culture systems. Methods Mol Biol, 2015. 1283: p. 147-59.
132. Fekete, N., et al., GMP-compliant isolation and large-scale expansion of bone marrow-derived MSC. PLoS One, 2012. 7(8): p. e43255.
133. Lange, C., et al., Accelerated and safe expansion of human mesenchymal stromal cells in animal serum free medium for transplantation and regenerative medicine. J Cell Physiol, 2007. 213(1): p. 18-26.
134. Tanthaisong, P., et al., Enhanced Chondrogenic Differentiation of Human Umbilical Cord Wharton's Jelly Derived Mesenchymal Stem Cells by GSK-3 Inhibitors. PLoS One, 2017. 12(1): p. e0168059.
135. Linares, G. R., et al., Preconditioning mesenchymal stem cells with the mood stabilizers lithium and valproic acid enhances therapeutic efficacy in a mouse model of Huntington's disease. Exp Neurol, 2016. 281: p. 81-92.
136. Ferensztajn-Rochowiak, E., et al., The effect of long-term lithium treatment of bipolar disorder on stem cells circulating in peripheral blood. World J Biol Psychiatry, 2017. 18(1): p. 54-62.
137. Ferensztajn-Rochowiak, E. and J. K. Rybakowski, The effect of lithium on hematopoietic, mesenchymal and neural stem cells. Pharmacol Rep, 2016. 68(2): p. 224-30.
138. Dong, B. T., et al., Lithium enhanced cell proliferation and differentiation of mesenchymal stem cells to neural cells in rat spinal cord. Int J Clin Exp Pathol, 2015. 8(3): p. 2473-83.
139. Zhu, Z., et al., Lithium stimulates human bone marrow derived mesenchymal stem cell proliferation through GSK-3beta-dependent beta-catenin/Wnt pathway activation. FEBS J, 2014. 281(23): p. 5371-89.
140. Tsoyi, K., et al., Carbon Monoxide Improves Efficacy of Mesenchymal Stromal Cells During Sepsis by Production of Specialized Proresolving Lipid Mediators. Crit Care Med, 2016. 44(12): p. e1236-e1245.
141. Ghanjati, F. and S. Santourlidis, Effect on Multipotency and Phenotypic Transition of Unrestricted Somatic Stem Cells from Human Umbilical Cord Blood after Treatment with Epigenetic Agents. Stem Cells Int, 2016. 2016: p. 7643218.
142. Dai, X., et al., Somatic nucleus reprogramming is significantly improved by m-carboxycinnamic acid bishydroxamide, a histone deacetylase inhibitor. J Biol Chem, 2010. 285(40): p. 31002-10.
143. Durcova-Hills, G., et al., Reprogramming primordial germ cells into pluripotent stem cells. PLoS One, 2008. 3(10): p. e3531.
144. Huh, Y. H., J. H. Ryu, and J. S. Chun, Regulation of type II collagen expression by histone deacetylase in articular chondrocytes. J Biol Chem, 2007. 282(23): p. 17123-31.
145. Wang, Z., et al., Infusion of Valproic Acid Into the Renal Medulla Activates Stem Cell Population and Attenuates Salt-Sensitive Hypertension in Dahl S Rats. Cell Physiol Biochem, 2017. 42(3): p. 1264-1273.
146. Lim, J., et al., Valproic acid enforces the priming effect of sphingosine-1 phosphate on human mesenchymal stem cells. Int J Mol Med, 2017. 40(3): p. 739-747.
147. Luo, Z., et al., Effects of histone deacetylase inhibitors on regenerative cell responses in human dental pulp cells. Int Endod J, 2017.
148. Hawkins, K. E., et al., Human Amniocytes Are Receptive to Chemically Induced Reprogramming to Pluripotency. Mol Ther, 2017. 25(2): p. 427-442.
149. Mahapatra, P. S., et al., Valproic acid assisted reprogramming of fibroblasts for generation of pluripotent stem cells in buffalo (Bubalus bubalis). Int J Dev Biol, 2017. 61(1-2): p. 81-88.
150. Dziadosz, M., et al., Effects of Pharmacological Agents on Human Amniotic Fluid-Derived Stem Cells in Culture. Stem Cells Dev, 2016.
151. Dehghan, S., et al., Oct4 transcription factor in conjunction with valproic acid accelerates myelin repair in demyelinated optic chiasm in mice. Neuroscience, 2016. 318: p. 178-89.
152. Petkov, S., et al., Long-Term Culture of Porcine Induced Pluripotent Stem-Like Cells Under Feeder-Free Conditions in the Presence of Histone Deacetylase Inhibitors. Stem Cells Dev, 2016. 25(5): p. 386-94.
153. Chen, X., et al., Valproic Acid Enhances iPSC Induction From Human Bone Marrow-Derived Cells Through the Suppression of Reprogramming-Induced Senescence. J Cell Physiol, 2016. 231(8): p. 1719-27.
154. Shi, H., et al., Roles of p53 and ASFJA in the Reprogramming of Sheep Kidney Cells to Pluripotent Cells. Cell Reprogram, 2015. 17(6): p. 441-52.
155. Gajzer, D., et al., Epigenetic and molecular signatures of cord blood CD34(+) cells treated with histone deacetylase inhibitors. Vox Sang, 2016. 110(1): p. 79-89.
156. Dehghan, S., et al., Exogenous Oct4 in combination with valproic acid increased neural progenitor markers: an approach for enhancing the repair potential of the brain. Life Sci, 2015. 122: p. 108-15.
157. Killer, M. C., et al., Immunosuppressive capacity of mesenchymal stem cells correlates with metabolic activity and can be enhanced by valproic acid. Stem Cell Res Ther, 2017. 8(1): p. 100.
158. Fila-Danilow, A., et al., The influence of TSA and VPA on the in vitro differentiation of bone marrow mesenchymal stem cells into neuronal lineage cells: Gene expression studies. Postepy Hig Med Dosw (Online), 2017. 71(0): p. 236-242.
159. Zhai, Y., et al., Histone deacetylase inhibitor valproic acid promotes the induction of pluripotency in mouse fibroblasts by suppressing reprogramming-induced senescence stress. Exp Cell Res, 2015. 337(1): p. 61-7.
160. Wang, J., et al., Identification of small activating RNAs that enhance endogenous OCT4 expression in human mesenchymal stem cells. Stem Cells Dev, 2015. 24(3): p. 345-53.
161. Wang, B. and Y. Hu, Promoter-Targeted Small Activating RNAs Alter Nucleosome Positioning. Adv Exp Med Biol, 2017. 983: p. 53-61.
162. Xu, C., et al., Long non-coding RNA GAS5 controls human embryonic stem cell self-renewal by maintaining NODAL signalling. Nat Commun, 2016. 7: p. 13287.
163. Hadjimichael, C., et al., MicroRNAs for Fine-Tuning of Mouse Embryonic Stem Cell Fate Decision through Regulation of TGF-beta Signaling. Stem Cell Reports, 2016. 6(3): p. 292-301.
164. Li, H. L., et al., miR-302 regulates pluripotency, teratoma formation and differentiation in stem cells via an AKT1/OCT4-dependent manner. Cell Death Dis, 2016. 7: p. e2078.
165. Sandmaier, S. E. and B. P. Telugu, MicroRNA-Mediated Reprogramming of Somatic Cells into Induced Pluripotent Stem Cells. Methods Mol Biol, 2015. 1330: p. 29-36.
166. Barta, T., et al., Brief Report: Inhibition of miR-145 Enhances Reprogramming of Human Dermal Fibroblasts to Induced Pluripotent Stem Cells. Stem Cells, 2016. 34(1): p. 246-51.
167. Muntoni, F. and M. J. Wood, Targeting RNA to treat neuromuscular disease. Nat Rev Drug Discov, 2011. 10(8): p. 621-37.
168. Chiriboga, C. A., et al., Results from a phase 1 study of nusinersen (ISIS-SMNRx) in children with spinal muscular atrophy. Neurology, 2016. 86(10): p. 890-7.
169. Hache, M., et al., Intrathecal Injections in Children With Spinal Muscular Atrophy: Nusinersen Clinical Trial Experience. J Child Neurol, 2016. 31(7): p. 899-906.
170. Meyer, K., et al., Improving single injection CSF delivery of AAV9-mediated gene therapy for SMA: a dose-response study in mice and nonhuman primates. Mol Ther, 2015. 23(3): p. 477-87.

1. A method of ameliorating the effects of spinal muscular atrophy comprising the steps of: a) identifying a subject suffering spinal muscular atrophy; b) providing a population of stem cells, and/or derivatives of stem cells; and b) administering said stem cells and/or derivatives of stem cells to said subject at a concentration and frequency sufficient to ameliorate the effects of spinal muscular atrophy.

2. The method of claim 1, wherein said ameliorated effects of spinal muscular atrophy are selected from the group consisting of: muscle strengthening, improved balance, improved fine motor skills, lessened tremors, improved appetite, improved ability to eat, improvement in walking.

3. The method of claim 2, wherein said spinal muscular atrophy is caused by mutations in the Survival Motor Neuron (SMN) gene.

4. The method of claim 3, wherein said mutations of said SMN gene is associated with reduction in SMN1 protein.

5. The method of claim 1, wherein said spinal muscular atrophy is selected from a group consisting of: a) Type 1 spinal muscular atrophy; b) Type 2 spinal muscular atrophy; c) Type 3 spinal muscular atrophy; and d) Type 4 spinal muscular atrophy.

6. The method of claim 1, wherein said stem cells are mesenchymal stem cells.

7. The method of claim 6, wherein said mesenchymal stem cells are plastic adherent.

8. The method of claim 6, wherein said mesenchymal stem cells positively express CD34.

9. The method of claim 6, wherein the mesenchymal stem cells are administered intravenously.

10. The method of claim 9, wherein the mesenchymal stem cells are administered a second time within 14 months of the first administration.

11. The method of claim 10, wherein the patient is administered the mesenchymal stem cells once or more per year.

12. The method of claim 6, wherein said mesenchymal stem cells are derived from tissues selected from the group consisting of: a) bone marrow; b) peripheral blood; c) adipose tissue; d) mobilized peripheral blood; e) umbilical cord blood; f) Wharton's jelly; g) umbilical cord tissue; h) skeletal muscle tissue; i) subepithelial umbilical cord; j) endometrial tissue; k) menstrual blood; and l) fallopian tube tissue.
